(19) United States
(12) Patent Application Publication
(10) Pub. No.: US 2011/0166555 A1
(43) Pub. Date: Jul. 7, 2011

(54) CARRIER FOR AN INSERTABLE MEDICAL DEVICE, INSERTION TOOLS, METHODS OF USE, AND KITS

(76) Inventors: Jianbo Zhou, Rancho Santa Margarita, CA (US); Signe R. Erickson, Menlo Park, CA (US); Krip Punja, Irvine, CA (US)

(21) Appl. No.: 12/895,092

(22) Filed: Sep. 30, 2010

Related U.S. Application Data
(60) Provisional application No. 61/247,127, filed on Sep. 30, 2009.

(51) Int. Cl. A61B 17/00; F16M 13/00; F16B 2/10; F16B 2/20

(52) U.S. Cl. 606/1; 248/274.1; 248/231.51

(57) ABSTRACT

Disclosed are device carriers for insertable medical devices. The device carrier holds an insertable medical device in a desired orientation to allow it to be grasped by an insertion tool. Additionally, the device carrier may be used to store and to protect the insertable medical device prior to the device being implanted at a target site in a patient. Also described are insertion tools that may be used with the device carrier for grasping the insertable medical device from the carrier, and inserting the medical device at the target site in the patient. Methods of using the device carriers and the insertion tools of the invention, and kits for practicing the methods of the invention are also disclosed.

### CROSS REFERENCE TO RELATED APPLICATIONS

This application claims priority to U.S. Provisional Application Ser. No. 61/247,127, filed Sep. 30, 2009, the disclosure of which is incorporated herein by reference.

### FIELD

The invention relates to carriers for holding insertable medical devices. The carrier holds the insertable medical device in a convenient orientation so that it can be grasped by an insertion tool. Also described are insertion tools for grasping the insertable medical device and for implanting the device into a patient. Also disclosed are methods of using the device carriers and insertion tools of the invention and kits for practicing the methods of the invention.

### BACKGROUND

Insertable medical devices of the type described in U.S. Pat. No. 6,719,750 (Varner et al.) and in U.S. Patent Application Publication No. 2005/0019371 are known for use in treating various disorders such as ophthalmic disorders. In many embodiments, these devices include a coil portion that contains one or more bioactive agents impregnated into a polymer. After insertion of the device, the bioactive agent elutes from the polymer to provide treatment at the implantation site. Although the polymer systems are durable, prior to implantation of the device, it is desirable to protect the polymer from contamination and/or damage that may occur if the polymer is not protected. Accordingly, what is desired is a carrier that securely holds the implantable device in a position that allows it to be readily grasped by the surgeon using an insertion tool.

### SUMMARY

The invention relates to device carriers for insertable medical devices. The device carrier holds an insertable medical device in a desired orientation to allow it to be grasped by an insertion tool. Additionally, the device carrier may be used to store and to protect the insertable medical device prior to the device being implanted at a target site in a patient. Also described are insertion tools that may be used with the device carrier for grasping the insertable medical device from the carrier, and inserting the medical device at the target site. In an exemplary embodiment, the insertable medical device is a polymer-coated coil that is impregnated with one or more bioactive agents. Prior to insertion of the insertable medical device into a patient, it is desired that the polymer-coated coil be protected from inadvertent contact that may potentially cause contamination or damage to the medical device.

Accordingly, in one aspect, the invention provides a device carrier for holding an insertable medical device in an orientation to allow the device to be grasped by an insertion tool, the device carrier comprising:

a top clamping member and a bottom clamping member that are connected for pivotal movement relative to one another;

a channel sized to receive a distal end of the insertion tool; wherein the channel is formed by a groove in the top clamping member and a groove in the bottom clamping member that align with one another to fowl the channel when the top clamping member and the bottom clamping member are in a closed position;

a top arm that extends from the top clamping member into the channel;

a bottom arm that extends from the bottom clamping member into the channel;

wherein the top arm and the bottom arm each contain a distal end that together hold the insertable medical device in the channel in an orientation for grasping by the insertion tool;

an alignment mechanism for rotationally aligning the insertion tool with the channel in an orientation that allows the insertable medical device to be grasped by the insertion tool without interference from the top and bottom arms;

a locking mechanism that holds the top clamping member and the bottom clamping member in the closed position wherein the locking member can be manually released to allow pivotal movement between the top and bottom clamping members;

wherein, when the locking mechanism is released, the top clamping member and the bottom clamping member can be pivoted between a closed position where the distal end of the top arm and the distal end of the bottom arm are proximate one another for holding the insertable medical device therebetween; and an open position where the top arm and the bottom arm are separated from one another in order to release the insertable medical device from the device carrier.

In another aspect, the invention provides an insertion tool for grasping an insertable medical device held in a device carrier, the insertion tool comprising: a handle; an actuating lever; an actuating mechanism; and a collet; wherein the actuating lever is operably connected to the collet by the actuating mechanism so that movement of the lever causes the collet to grasp the insertable medical device; and wherein the distal end of the insertion tool comprises an alignment mechanism for orienting the insertion tool relative to a device carrier.

In yet another aspect, the invention provides a method for grasping an insertable medical device from a device carrier with an insertion tool, the method comprising the steps of
(A) providing a device carrier of the invention as described herein wherein the device carrier contains an insertable medical device;
(B) providing an insertion tool of the invention as described herein;
(C) aligning the insertion tool with the device carrier using the alignment mechanism;
(D) inserting the distal end of the insertion tool into the channel of the device carrier;
(E) moving the actuating lever of the insertion tool in order to cause the collet of the insertion tool to grasp the insertable medical device; and
(F) pivoting the top clamping member and the bottom clamping member of the device carrier relative to one another to release the insertable medical device from between the top and bottom arms of the device carrier.

In yet another aspect, the invention provides a kit for practicing the method of the invention, the kit comprising: (a) a device carrier of the invention as described herein; and (b) an insertion tool of the invention as described herein.

### BRIEF DESCRIPTION OF THE FIGURES

FIG. 1 is an illustration of an insertable medical device having a helically shaped body member and a head, and which can be used in conjunction with the device carrier and insertion tool of the invention.

FIGS. 1A-1C show various alternate embodiments of the head of the insertable medical device of FIG. 1.

FIG. 2 is cross-sectional view of an eye showing a typical positioning of the insertable medical device of FIG. 1 in an eye of a patient.

FIG. 3 is a perspective view of an embodiment of an insertion tool suitable for use with the device carrier of the invention.

FIG. 4 is a longitudinal cross-sectional view of the insertion tool of FIG. 3.

FIG. 5 is a longitudinal cross-sectional view of the insertion tool of FIG. 3 with the collet extended for receiving an insertable medical device such as shown in FIG. 1.

FIG. 6 is perspective view of a device carrier of the invention

FIG. 7 is an end view of the device carrier of FIG. 6.

FIG. 8 shows a device carrier of the invention that is mounted on a primary carrier.

FIG. 9 shows exemplary packaging scheme for a device carrier of the invention.

FIGS. 10A-10E show a device carrier of the invention being used by a surgeon according to the method of the invention.

### DETAILED DESCRIPTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

As background for describing the device carrier of the invention, an insertable medical device of the type suitable for use with the device carrier and insertion tool of the invention is described below.

The device carriers of the invention are useful for holding an insertable medical device. As used herein the term “insertable medical device” refers generally to a medical article that can be held by an insertion tool and can be inserted into a target site (e.g., eye) of a patient for delivery of one or more therapeutic agents and/or medicaments. An exemplary insertable medical device that is designed to be inserted into a limited access region of the body is described in U.S. Pat. No. 6,719,750 (Varner et al.) and in U.S. Patent Application Publication No. 2005/0019371 A1. These patent documents describe various non-linear devices that can be inserted into a target site and may be used to deliver therapeutic agents and/or medicaments from the device. In some embodiments of these documents, the device includes a portion that has a substantially coiled or helical configuration that can be introduced into the target site during insertion.

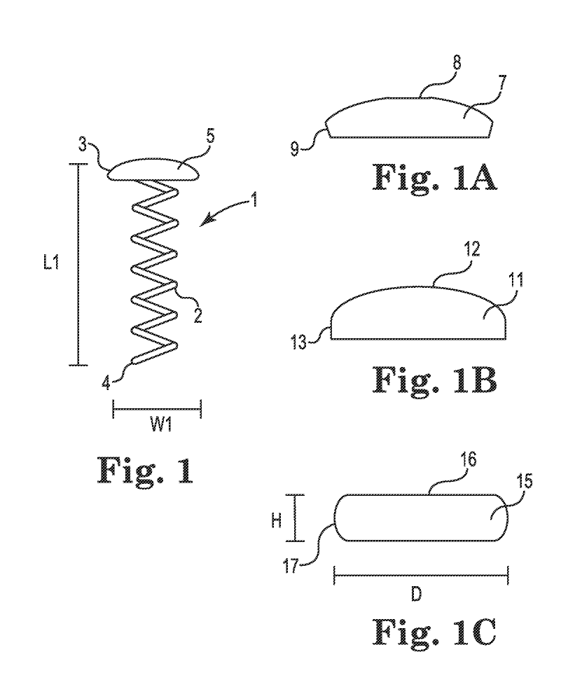

Referring now to FIG. 1, an embodiment of the helically shaped device of U.S. Pat. No. 6,719,750 is shown. As shown in FIG. 1, an insertable medical device 1 includes a non-linear shaped body member 2, a proximal end 3, and a distal end 4. Preferably, the body member 2 has a substantially coiled or helical shape. The coil shape of the body member allows the device to be screwed or twisted into the target site, such as the eye, through an insertion in a portion of the eye, such as the sclera. The distal end 4 of the body member 2 can have a blunt or non-blunt shape. In some embodiments, the distal end has a non-blunt shape and is therefore configured to allow tissue to be pierced during the insertion of the device. For example, the distal end 4 of the device can have a pointed or beveled ramp-like configuration useful for piercing the eye during insertion. In one embodiment, the pointed or beveled ramp-like configuration has a ramp-like angle of about 30°. If the distal end 4 of the body member 2 is used to pierce the eye during insertion, at least the distal end 4 is fabricated of a rigid, non-pliable material suitable for piercing the eye. Such materials are well known and may include, for example, biocompatible metals and polymers.

FIG. 1 also shows the insertable medical device 1 having a head 5 located at the proximal end 3 of the device 1. The head 5 can be of any suitable configuration and size for insertion of the device and/or placement of the device into a target site. In choosing or forming a head of a particular size and configuration (geometry), the head typically has the inverse geometry of that of the socket of the securing member.

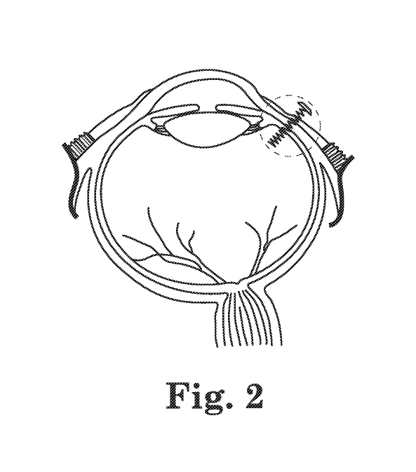

The head can also function to stabilize the device once implanted into a target site, such as the eye (see, FIG. 2). For example, the device can be inserted into the vitreous of the eye through a penetration or incision in the scleral tissue until the distal face of the head abuts the scleral tissue. If desired, the head may then be sutured to the eye, using one or more holes that can be optionally present in the head, to further stabilize and prevent the device from moving once it is implanted in its desired location.

The overall size and shape of the head is not limited to any particular configuration. In most embodiments, when viewed from the proximal end of the device, the head will have a circular shape, and generally a cap-like shape when viewed in perspective. Referring to FIG. 1, although the head 5 is shown to have a generally smooth cap-like shape, the head may optionally have a faceted dome-like shape. Alternatively, when viewed from the proximal end of the device, the head may have non-circular shape, for example, a triangular, rectangular, hexagonal, etc., shape. However, to minimize irritation to the eye, the head preferably has a rounded surface. In some aspects, the head has a cap or rim configuration similar to or the same as that shown in FIG. 1A. The head 7 includes a flat top 8 at the proximal end of the medical device, and a straight wall 9 about periphery of the head 7.

In some aspects, the head has a configuration similar to or the same as that shown in FIG. 1B. The head 11 includes a rounded top 12 at the proximal end of the device, and a straight wall 13 about periphery of the head 11. In some aspects, the head has configuration similar to or the same as that shown in FIG. 1C. The head 15 includes a flat top 16 at the proximal end of the device, and a rounded wall 17 about periphery of the head 15.

In many aspects, the head of the medical device is small and has a displacement volume of about 5 mm³ or less. In one exemplary design the head has a displacement volume of about 2 mm³ or about 2.5 mm³. In some aspects, for example, referring to FIG. 1, the head has a diameter (D) of about 2.5 mm or less. In one exemplary design the head has a diameter (D) of about 2.0 mm. In some aspects, the head has a height (H) of about 0.5 mm or less. In one exemplary design the head has a height (H) of about 0.38 mm.

In some embodiments, the head on the medical device has few or no indentations or recesses, and is therefore substantially smooth. In some aspects, a medical device having a head with this configuration may be preferred, as tissue, which may otherwise in-grow into these indentations or recesses, can be prevented.

In other aspects the head on the medical device has one, and preferably two or more indentations or recesses. The indentations or recesses can be useful for stabilizing the medical device in the securing member, which can be provided by the securing member having one or more posts configured for insertion in the indentations or recesses. Upon insertion of the medical device, the indentations or recesses can be filled in with a sealant to form a substantially smooth surface.

Preferably, the head of the insertable medical device is configured to remain outside the eye and, as such, the head is sized so that it will not pass into the eye through the opening in the eye through which the device is inserted (see, FIG. 2). As indicated, the head may further be designed such that it can be secured to the surface surrounding the insertion.

The materials used in fabricating the insertable medical device are not particularly limited. In some embodiments these materials are biocompatible and preferably insoluble in body fluids and tissues the device comes into contact with. Further, it is preferred that the device is fabricated of a material that does not cause irritation to the portion of the eye that it contacts. In some aspects the insertable medical device is fabricated from a metal or alloy. Metals that can be used to fabricate the device include platinum, gold, or tungsten, as well as other metals such as rhenium, palladium, rhodium, ruthenium, titanium, nickel, and alloys of these metals, such as stainless steel, titanium/nickel, nitinol alloys, cobalt chrome alloys, non-ferrous alloys, and platinum/iridium alloys. One exemplary alloy is MP35N. Other materials include ceramics. The ceramics include, but are not limited to, silicon nitride, silicon carbide, zirconia, and alumina, as well as glass, silica, and sapphire. Polymeric materials can also be used to fabricate the device. Exemplary polymeric material materials can be pliable and include, by way of example, silicone elastomers and rubbers, polyolefins, polyurethanes, acrylates, polycarbonates, polyamides, polyimides, polyesters, and polysulfones.

The dimensions and configurations of the insertable medical device can depend on the application of the device. When a device such as shown in FIG. 1 is used to deliver substances to the posterior chamber of the eye, the device is preferably designed for insertion through a small incision that requires few or no sutures for scleral closure, after the insertion procedure has be completed. As such, the device is preferably inserted through an incision that is no more than about 1 mm in cross-section, for example, ranging from about 0.25 mm to about 1 mm in diameter, more preferably less than 0.5 mm in diameter. Accordingly, the cross-section of the tube or wire forming the body member 2 is preferably no more than about 1 mm in diameter, with a preferred range from about 0.25 mm to about 1 mm in diameter. More preferably, the cross section is no greater than 0.5 mm in diameter. As shown in FIG. 1, the non-linear body member 2 is cylindrical in shape, with a circular cross-section. However, the shape of the body member is not limited and, for example, may alternatively have square, rectangular, octagonal or other cross-sectional shapes. If the material (such as a tube or wire) forming the body member 2 is not cylindrical, the largest dimension of the cross section can be used to approximate the diameter.

When used to deliver agents to the posterior chamber of the eye, the body member 2 has a length (L1) from its proximal end to its distal end 6. The length (L1) can be less than about 1.5 cm, or less than 1.0 cm, and preferably in the range from about 0.25 cm to about 1.0 cm. The length (L1) can be such that when the distal portion of the head 5 abuts the outer surface of the eye, the proximal portion of the body member is positioned within the posterior chamber of the eye.

In many embodiments, the device carrier of the invention is used in cooperation with an insertion tool. The insertion tool is designed to grasp or dock with the insertable medical device that is initially held in the device carrier. The insertion tool can then be used to insert the insertable medical device at a desired treatment site of a patient (e.g., an eye).

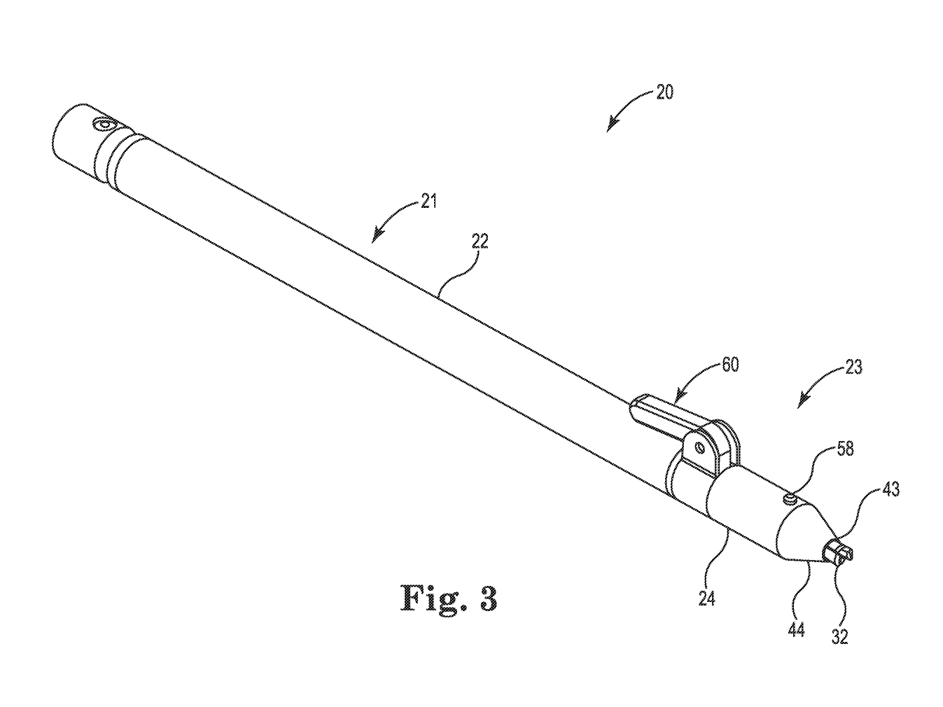

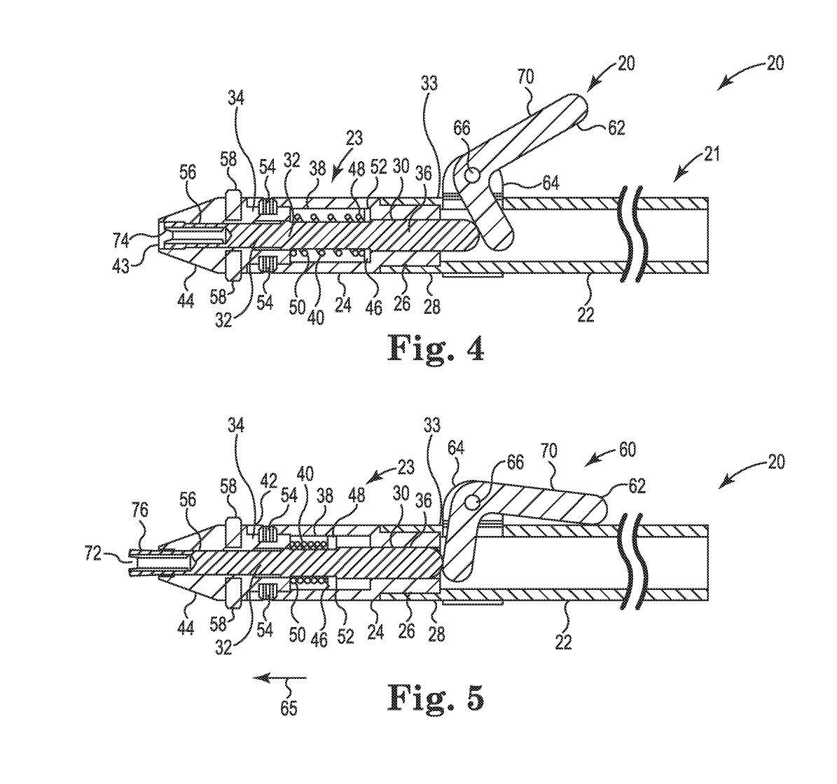

An embodiment of an insertion tool that is suitable for use with an insertable medical device and device carrier of the invention will now be described with reference to FIGS. 3-4. FIG. 3 shows a perspective view of one embodiment of an insertion tool 20. Insertion tool 20 includes proximal portion 21 and distal portion 23. In some embodiments, as shown in FIG. 3, the proximal portion 21 is configured to be manually held and operated by a user, typically a surgeon. Therefore, the proximal portion 21 of the insertion tool 20 can be configured as a handle 22 having any shape that is suitable for performing the insertion process. For example, the handle may have a simple cylindrical shape or, alternatively, may have a shape that is designed to ergonomically fit portions of the surgeon's hand. For example, the handle can have raised portions that allow the surgeon to have a better grip of the insertion tool using fingers. The handle may also have a textured or patterned surface to reduce slippage or increase frictional forces between the user and the instrument. This sort of surface can be useful if the user is wearing a hand covering, such as latex gloves.

The proximal portion 21 can be fabricated from any suitable material, including plastics, composites, ceramics, metals, and metal alloys. In some cases it is preferred to use a material that can be readily sterilized, for example, by heat and/or pressure sterilization, such as autoclaving, or by irradiation, such as gamma irradiation, or by chemical sterilization, such as ethylene oxide sterilization. In other cases the handle can be disposable by fabricating all or parts of handle with plastic materials, such as ABS, Teflon™, and Delrin™. The handle can be of any suitable length and outer diameter for use. For example, a handle having a length in the range of about 10 cm or 11 cm can be particularly useful when utilized in methods of rotatably inserting an insertable medical device into a target site (e.g., an eye).

The distal portion 23 includes housing 24, nose piece 44, and actuating mechanism 60. The nose piece 44 has opening 43 to allow the distal portion of collet 32 to travel into and out of the nose piece 44 during operation of the insertion tool 20 in response to actuating mechanism 60. Referring now to FIG. 4, a longitudinal cross-sectional view of the insertion tool 20 of FIG. 3 is shown. As shown in FIG. 4, the proximal portion 21 of the insertion tool 20 includes handle 22. Handle 22 is attached to housing 24. In the embodiment of FIG. 4, the housing 24 includes a treaded portion 26 that fits within a threaded portion 28 of handle 22 to form a connection between the housing 24 and the handle 22. Housing 24 includes an axial bore 30 that extends from opening 33 to opening 34. The axial bore 30 has first length 36 that has a diameter that is sized to allow sliding movement of collet 32. Axial bore 30 has second length 38 that has a diameter that is larger than the diameter of first segment 30. The second length 38 of axial bore 30 holds compression spring 40, which is positioned around the outer surface of collet 32. Axial bore 30 has third length 42 which is sized appropriately to receive nose piece 44. As shown in FIG. 4, a retaining washer 46 is fitted around collet 32 at one end of compression spring 40. Retaining washer 46 has circular opening 48 with a diameter that is sized appropriately to fit over the middle portion 50 of collet 32 and rest on shoulder 52. In this way, when collet 32 is advanced longitudinally, shoulder 52 transmits the longitudinal movement to washer 46 that in turn causes compression spring 40 to be compressed.

In the embodiment shown in FIG. 4, housing 24 is connected to the nose piece 44 with set screws 54. It is understood that other forms of attachment may also be used. Nose piece 44 includes an axial bore 56 that is aligned with the axial bore 30 in housing 24 and is sized for sliding movement of collet 32. Nose piece 44 includes alignment splines 58 which extend radially outward from the nose piece 44. The alignment splines 58 are typically fitted into the nose piece 44 on opposite sides of the nose piece, for example, at 180° from each other. Other orientations (e.g., 90°) may also be useful. The alignment splines 58 provide an alignment mechanism for precise orientation of the insertion tool 20 relative to the device carrier for grasping the insertable medical device.

Collet 32 includes longitudinal fingers 76 that extend at the distal end 74 of the collet to define cavity 72. Between the fingers 76 are longitudinal slits. The longitudinal slits along with the resilient nature of the material foaming the collet 32 allow the fingers 76 to be compressed radially inward to grasp the head of an insertable medical device, such as the insertable medical devices described hereinabove. The fingers 76 of collet 32 may contain arcuate regions on the inner portion of the fingers that facilitate grasping the head of the insertable medical device, The arcuate regions may be shaped, for example, to match the contour of the head of an insertable medical device to be grasped.

Referring now to FIG. 5, the handle 22 portion of insertion tool 20 includes an actuating mechanism 60 that allows the user to move the collet 32 longitudinally along the axial bore 30 and as it extends through the housing 24 and nose piece 44. Actuating mechanism 60 includes lever 62 that is mounted on bracket 64 with pin 66 so that lever 62, when depressed by the surgeon, can pivot at pin 66.

In operation of insertion tool 20, handle 22 of insertion tool 20 is held in the hand of a surgeon. When the surgeon desired to grasp an insertable medical device, the surgeon presses down on surface 70 of lever 62. Pressing down on surface 70 of lever 62 causes lever 62 to pivot as shown in FIG. 5. Pivoting of lever 62 causes lever 62 to push collet 32 in a longitudinal direction as shown by arrow 65 though bore 30 so that the distal end 74 of collet 32 emerges from opening 43 in nose piece 44 as shown in FIG. 5. When the distal end 74 of the collet 32 emerges from opening 43 in nose piece 44, the fingers 76 on collet 32 are allowed to radially expand, and the insertion tool 20 is prepared to receive the head of an insertable medical device. The surgeon can then position the cavity 72 of collet 32 over the head of an insertable medical device so that fingers 76 are in a position to grasp the head of the insertable medical device. The lever 62 is then released causing compression spring 40 to push collet 32 longitudinally in the reverse direction of arrow 65 causing the fingers 76 of collet 32 to radially contract upon entering bore 56 of nose piece 44. The radial contraction of the fingers 76 of collet 32 causes insertion tool 20 to securely grip the head of the insertable medical device.

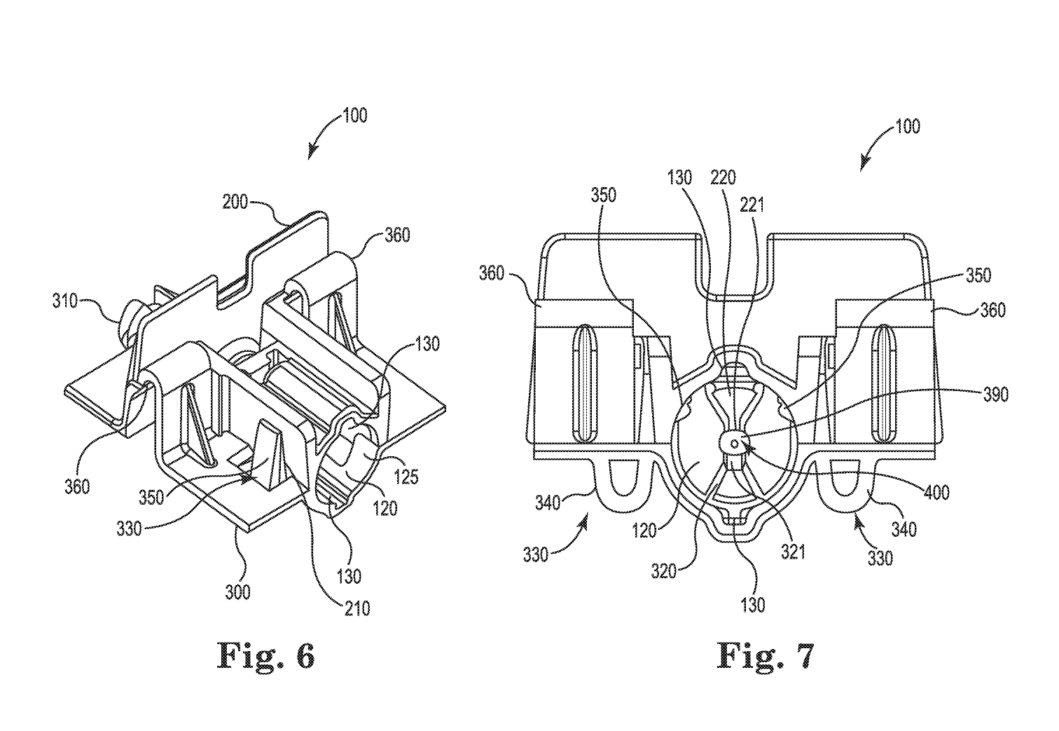

Referring now to FIG. 6, a perspective view of an insertable medical device carrier 100 is shown. Device carrier 100 includes top clamping member 200 and bottom clamping member 300. Top clamping member 200 and bottom clamping member 300 are connected for pivotal movement relative to one another. In the embodiment of FIG. 6, the pivotal movement is provided by hinge 310. Hinge 310 allows top clamping member 200 and bottom clamping member 300 to move relative to one another in a clamshell-like opening and closing motion when the device carrier 100 is in use. The hinge 310 may take any desired form that is convenient for manufacturing or for other purposes. In an exemplary embodiment, the hinge comprises a thin portion of a polymer material (i.e. a living hinge) that connects top clamping member 200 to bottom clamping member 300. The thinned portion preferentially bends creating hinging action when top clamping member 200 and bottom clamping member 300 are moved relative to one another. Top clamping member 200 and bottom clamping member 300 have arcuate channels that together form channel 120 having opening 125 when the device carrier is closed. Opening 125 and channel 120 are sized for receiving the distal end of an insertion tool as described herein. Channel 120 includes grooves 130 that extend longitudinally down channel 120 on opposite sides. The longitudinal grooves 130 are used for orienting the insertion tool when it is inserted into channel 120. More specifically, the alignment pins 58 of the insertion tool 20 fit within the longitudinal grooves 130 thereby orienting the insertion tool 20 to grasp the insertable medical device.

Referring now to FIG. 7, the top clamping member 200 and bottom clamping member 300 of device carrier 100 each include an arm 220 and 320, respectively, that function to position and hold the head 390 of an insertable medical device (as previously described herein with reference to FIG. 1) in an orientation that holds the head 390 of the insertable medical device in channel 120 so that it can be grasped with an insertion tool that is inserted into the channel 120. More specifically, when the device carrier is in a closed position, the distal end 221 of top arm 220 and the distal end 321 of bottom arm 320 together securely hold the head 390 of the insertable medical device with the coil (not shown) of the device extending longitudinally down channel 120 away from opening 125. Together, the distal end 221 and 321 are in contact with the head 390 of the insertable medical device only around a portion of the outer circumference of the head, for example, about 90 degrees or less. This allows access to the remaining portion of the head for grasping by the collet 32 of the insertion tool 20 (see, FIG. 3).

As shown in FIGS. 6-7, device carrier 100 typically includes one or more locking mechanisms in order to maintain the device carrier 100 in a closed and locked position until such time as the insertable medical device has been grasped by the insertion tool. By “locked” it is meant that top clamping member 200 and bottom clamping member 300 of the device carrier cannot be pivoted at hinge 310. The primary locking mechanism of device carrier 100 includes two hooks 360 that extend from bottom clamping member 300 and engage holding tabs 370 on top clamping member 200. As shown in FIGS. 6-7, hooks 360 engage holding tabs 370 on lever surface 680 in order to hold device carrier 100 in a closed position. Opening of device carrier 100 can be achieved by applying pressure to lever surface 680, for example, by applying finger pressure with the surgeon's thumb, as will be described in more detail below.

In some embodiments, the bottom clamping member 300 of device carrier 100 includes secondary locking tabs 330. In many embodiments, the secondary locking tabs 330 include U-shaped hinge portions 340 and locking end tabs 350. When device carrier 100 is in a closed position, end tabs 350 engage openings 210 in top clamping member 200 causing the top clamping member 200 to be locked to the bottom clamping member 300. As shown in FIG. 7, when device carrier 100 is in a closed and locked position, end tabs 350 of secondary locking tabs 330 extend into channel 120. When in use, the distal end of an insertion tool (see, FIGS. 3-4) is inserted into channel 120. Insertion of the distal end of the insertion tool into channel 120 causes end tabs 350 to retract thereby unlocking top clamping member 200 from bottom clamping member 300. In this way, the device carrier 100 remains locked until such time as the insertion tool is inserted into channel 120.

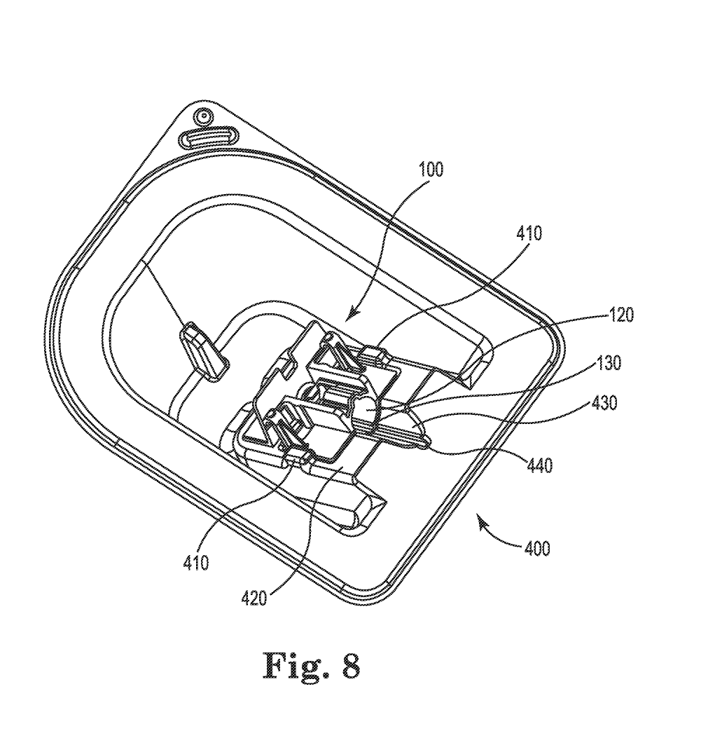

Referring now to FIG. 8, an embodiment of a device carrier of the invention that is affixed to a primary packaging tray is shown. In FIG. 8, device carrier 100 is affixed to the bottom surface 420 of a primary packaging tray 405. As shown in FIG. 8, tray 405 includes mounting clips 410 that are positioned for mounting the device carrier 100 to the bottom surface 420 of tray 405. Molded into the bottom surface 420 of tray 405 is groove 430. Grove 430 coincides with channel 120 in device carrier 100 in order to assisting the surgeon in guiding the insertion tool 20 (see, FIG. 3) into opening 125 of device carrier 100. Along the bottom surface of groove 430 an alignment groove 440 is molded into the tray 405. Alignment groove 440 is aligned with the sidewall slot 130 that is present on bottom clamping member 300 (see, FIG. 7) of device carrier 100. As discussed above, alignment groove 440 along with alignment grooves 130 on device carrier 100 assists the surgeon in radially orienting the insertion tool 20 so that that the collet 32 is aligned to grasp the head 390 of the insertible medical device 400.

Materials used in fabricating the device carrier of the invention typically include polymers that are capable of being injection molded or thermoformed. Examples of suitable polymers for the injection molded device carrier include polycarbonate, polypropylene, polymethyl methacrylate, styrene, methyl methacrylate, polybutylene, and terephthalate. A preferred material for forming the device carrier is polypropylene.

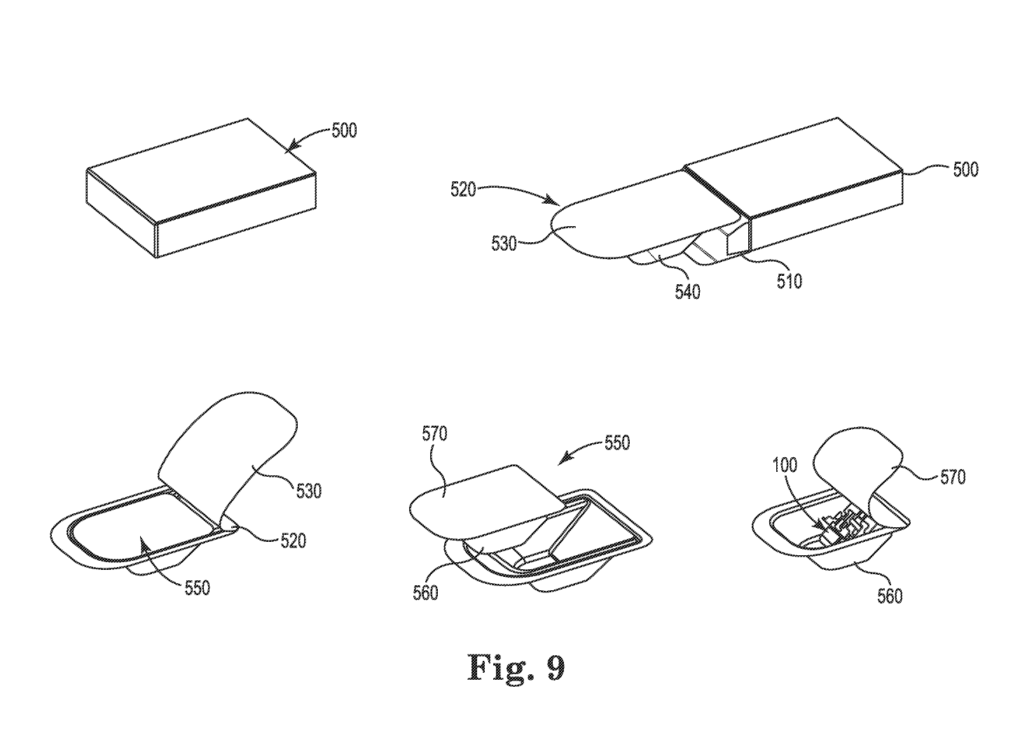

Referring now to FIG. 9, an example of a finished product-packaging configuration for the device carrier 100 is shown. As a shipping and labeling container, the device carrier 100 of the invention can be packaged, for example, in outer cardboard box 500 or other disposable container. As shown in Step 2 of FIG. 9, box 500 can be opened at flap 510 to allow access to a sealed secondary package 520. The sealed secondary package 520 includes removable lid 530 and tray 540. The removable lid 530 may be formed, for example, from Tyvek brand polyolefin film or other suitable flexible film. Other lid materials may also be used. As shown in Step 3, upon removal of the lid 530, the primary package 550 can be removed from the secondary package 520. As shown in Step 4, the primary package 550 includes tray 560 and removable lid 570. The removable lid 570 may be formed, for example, from Tyvek brand polyolefin film or other suitable flexible film. As shown in Step 5, the removable lid 570 can be removed from the primary package 550 to allow access by the surgeon to the device carrier 100 that is affixed to the tray 560.

The primary and secondary trays are typically thermoformed from polymers that are capable of being injection molded or thermoformed. Examples of suitable polymers for the primary and secondary tray include, polyvinyl chloride, polystyrene, and polyethylene, and polyethylene terephthalate glycol.

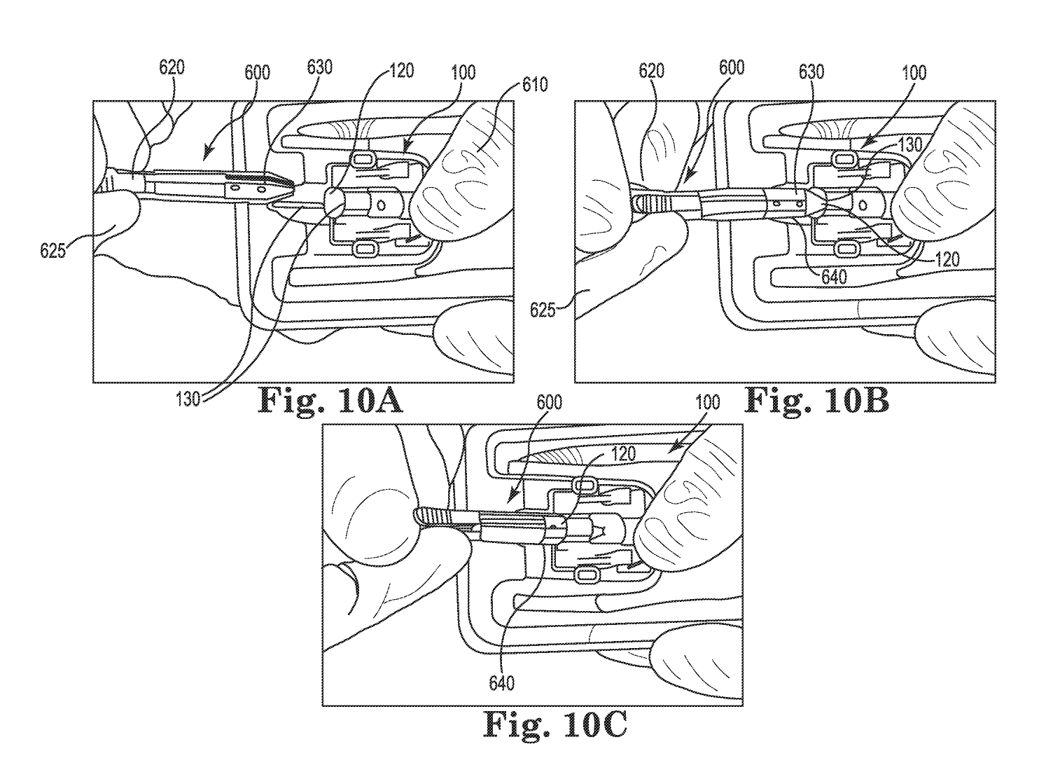

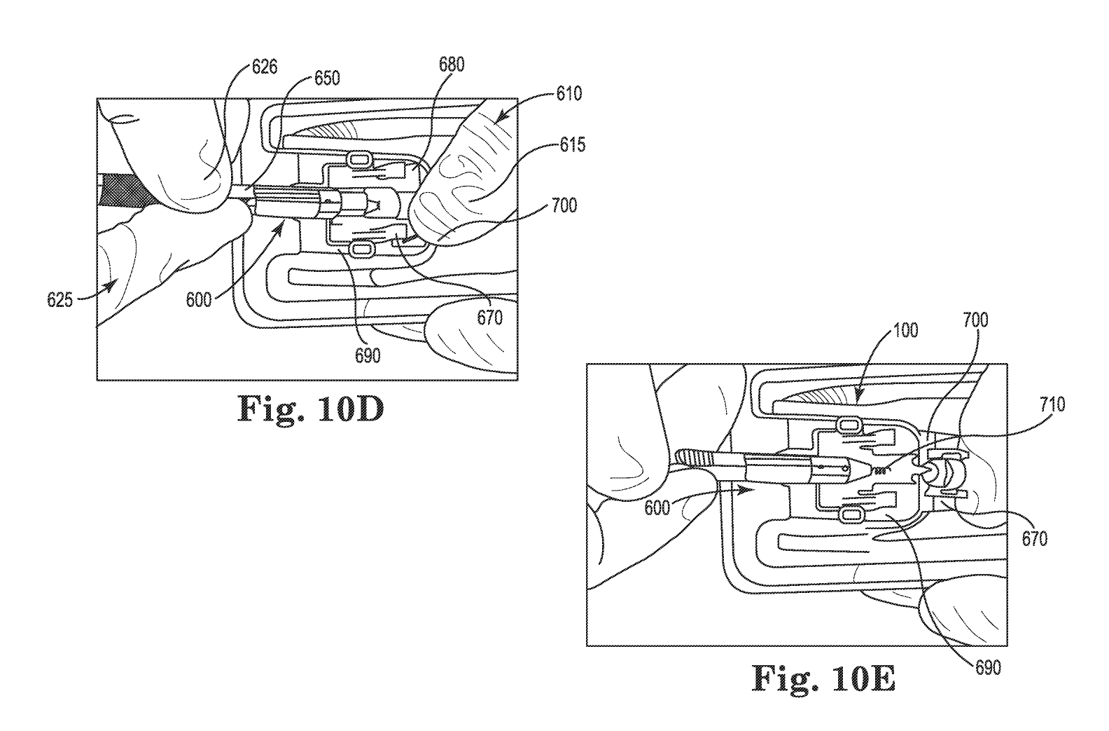

Referring now to FIGS. 10A-10E, the use of the device carrier 100 of the invention is exemplified. Referring now to FIG. 10A, an embodiment of device 100 is shown being prepared for use by a surgeon. As shown in FIG. 10A, the surgeon grasps the device 100 with left hand 610 and grasps the proximal end 620 of insertion tool 600 with right hand 625. Insertion tool 600 includes alignment pins 630 positioned at 180 degrees relative to one another on distal end 640 of insertion tool 600. Referring now to FIG. 10B, the surgeon is shown preparing to insert the distal end 640 of insertion tool 600 into opening 125 in device carrier 100. To prepare for insertion, the alignment pins 630 on insertion tool 600 are positioned so that they are aligned with the sidewall slots 130 of channel 120 in device carrier 100.

Referring now to FIG. 10C, the surgeon has inserted the distal end 640 of insertion tool 600 into channel 120 in device carrier 100 so that the distal end 640 comes in contact with the head of an insertable medical device (not shown in FIG. 10C) that is mounted in device carrier 100. Inserting the insertion tool 600 into the channel 120 of device carrier 100 causes the secondary locking tabs 350 (see, FIGS. 6-7) to retract. In the next step, as shown in FIG. 10D, the surgeon uses the thumb 626 of his right hand 625 to depress the actuator 650 of insertion tool 600. Depressing the actuator 650 causes the collet 32 (see, FIG. 3) of the insertion tool 600 to grasp the head 5 (see, FIG. 1) of the insertable medical device (not shown in FIG. 10D). As shown in FIGS. 10D-10E, once grasped, the surgeon releases the actuator 650 and uses the thumb 615 of his left hand 610 to put pressure against surface 680 causing top clamping member 670 of device carrier 100 to pivot away from bottom clamping member 690 at hinge 700. Upon pivoting, the insertable medical device 710 is released from the device carrier 100. FIG. 10E shows device carrier 100 in an opened state showing insertable medical device 710 held by insertion tool 600. The insertable medical device 710 is now prepared for insertion into a patient at a desired treatment site by the surgeon according to known techniques.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention which is indicated by the following claims.

What is claimed is:

1. A device carrier for holding an insertable medical device in an orientation to allow the device to be grasped by an insertion tool, the device carrier comprising:
a top clamping member and a bottom clamping member that are connected for pivotal movement relative to one another;
a channel sized to receive a distal end of the insertion tool; wherein the channel is formed by a groove in the top clamping member and a groove in the bottom clamping member that align with one another to form the channel when the top clamping member and the bottom clamping member are in a closed position;
a top arm that extends from the top clamping member into the channel;
a bottom arm that extends from the bottom clamping member into the channel;
wherein the top arm and the bottom arm each contain a distal end that together hold the insertable medical device in the channel in an orientation for grasping by the insertion tool;
an alignment mechanism for rotationally aligning the insertion tool with the channel in an orientation that allows the insertable medical device to be grasped by the insertion tool without interference from the top and bottom arms;
a locking mechanism that holds the top clamping member and the bottom clamping member in the closed position wherein the locking member can be manually released to allow pivotal movement between the top and bottom clamping members;
wherein, when the locking mechanism is released, the top clamping member and the bottom clamping member can be pivoted between a closed position where the distal end of the top arm and the distal end of the bottom arm are proximate one another for holding the insertable medical device therebetween; and an open position where the top arm and the bottom arm are separated from one another in order to release the insertable medical device from the device carrier.

2. The device carrier of claim 1, further including an insertable medical device held between the top arm and the bottom arm of the device.

3. The device carrier of claim 2, wherein the insertable medical device comprises a body member that extends from a head; and wherein the distal ends of the top arm and the bottom arm of the device carrier together hold the head of the insertable medical device in the channel with the body member extending longitudinally in the channel.

4. The device carrier of claim 1, further including a second locking tab that locks the top clamping member of the device carrier to the bottom clamping member of the device carrier so that the top clamping member and the bottom clamping member cannot be pivoted; wherein the secondary locking tab includes an actuating mechanism including an actuator that extends into the channel of the device carrier; wherein the second tab can be actuated by inserting the insertion tool into the channel; and wherein, when released, the top clamping member and the bottom clamping member can be pivoted between a closed position where the distal end of the top arm and the distal end of the bottom arm are proximate one another for holding the insertable medical device therebetween; and an open position where the top arm and the bottom arm are separated from one another in order to release the insertable medical device from the device carrier.

5. The device carrier of claim 1, wherein the alignment mechanism comprises one or more grooves in the channel for mating with one or more splines on the insertion tool;
wherein the grooves in the channel are configured so that upon inserting the insertion tool into the channel the insertion tool is aligned with the device carrier to allow the insertable medical device to be grasped by the insertion tool without interference from the top and bottom aims.

6. The device carrier of claim 5, wherein the one or more grooves are aligned with the top and bottom arms.

7. The device carrier of claim 1, wherein the top clamping member and the bottom clamping member are connected for pivotal movement by a hinge.

8. The device carrier of claim 8, wherein the hinge comprises a thin bendable portion of a polymer material.

9. The device carrier of claim 1, wherein the device carrier comprises a polymer selected from the group consisting of polycarbonate, polypropylene, polymethyl methacrylate, styrene, methyl methacrylate, polybutylene, and terephthalate.

10. The device carrier of claim 1, wherein the device carrier is affixed to a primary packaging tray.

11. The device carrier of claim 10, wherein the device carrier is affixed to the primary packaging tray with one or more mounting clips.

12. The device carrier of claim 10, wherein the primary packaging tray includes a groove in a bottom surface thereof; and wherein the groove coincides with the channel of the device carrier.

13. An insertion tool for clamping onto an insertable medical device held in a device carrier, the insertion tool comprising: a handle; an actuating lever; an actuating mechanism; and a collet; wherein the actuating lever is operably connected to the collet by the actuating mechanism so that movement of the lever causes the collet to grasp the insertable medical device; wherein the distal end of the insertion tool comprises an alignment mechanism for orienting the insertion tool with respect to a device carrier.

14. The insertion tool of claim 13, wherein the collet includes at least two longitudinally extending fingers that are spaced radially at a distance from one another.

15. The insertion tool of claim 13, wherein the insertion tool includes a collet having and end opening for receiving the insertable medical device; and wherein the collet comprises two longitudinally extending fingers positioned radially at about 180° from one another around the end opening.

16. The insertion tool of claim 14, wherein the fingers have arcuate regions that are shaped to match the contour of a head of an insertable medical device.

17. A method for grasping an insertable medical device from a device carrier with an insertion tool, the method comprising the steps of:
(A) providing a device carrier comprising:
a top clamping member and a bottom clamping member that are connected for pivotal movement relative to one another;
a channel sized to receive a distal end of the insertion tool; wherein the channel is formed by a groove in the top clamping member and a groove in the bottom clamping member that align with one another to form the channel when the top clamping member and the bottom clamping member are in a closed position;
a top arm that extends from the top clamping member into the channel;
a bottom arm that extends from the bottom clamping member into the channel;
wherein the top arm and the bottom arm each contain a distal end that together hold the insertable medical device in the channel in an orientation for grasping by the insertion tool;
an alignment mechanism for rotationally aligning the insertion tool with the channel in an orientation that allows the insertable medical device to be grasped by the insertion tool without interference from the top and bottom arms;
a locking mechanism that holds the top clamping member and the bottom clamping member in the closed position wherein the locking member can be manually released to allow pivotal movement between the top and bottom clamping members;
wherein, when the locking mechanism is released, the top clamping member and the bottom clamping member can be pivoted between a closed position where the distal end of the top arm and the distal end of the bottom arm are proximate one another for holding the insertable medical device therebetween; and an open position where the top arm and the bottom arm are separated from one another in order to release the insertable medical device from the device carrier;
wherein the device carrier further includes an insertable medical device having a head wherein the insertable medical device is held between the top arm and the bottom arm of the device;
(B) providing an insertion tool comprising: a handle; an actuating lever; an actuating mechanism; and a collet; wherein the actuating lever is operably connected to the collet by the actuating mechanism so that movement of the lever causes the collet to grasp the insertable medical device; wherein the insertion tool further includes an alignment mechanism for orienting the insertion tool with respect to the device carrier;
(C) aligning the insertion tool with the device carrier using the alignment mechanism;
(D) inserting the distal end of the insertion tool into the channel of the device carrier;
(E) moving the actuating lever of the insertion tool in order to cause the collet to grasp the insertable medical device; and
(F) pivoting the top clamping member and the bottom clamping member relative to one another to release the insertable medical device from between the top and bottom arms of the device carrier.

18. A kit comprising:
(A) a device carrier comprising:
a top clamping member and a bottom clamping member that are connected for pivotal movement relative to one another;
a channel sized to receive a distal end of the insertion tool; wherein the channel is formed by a groove in the top clamping member and a groove in the bottom clamping member that align with one another to form the channel when the top clamping member and the bottom clamping member are in a closed position;
a top arm that extends from the top clamping member into the channel;
a bottom arm that extends from the bottom clamping member into the channel;
wherein the top arm and the bottom arm each contain a distal end that together hold the insertable medical device in the channel in an orientation for grasping by the insertion tool;
an alignment mechanism for rotationally aligning the insertion tool with the channel in an orientation that allows the insertable medical device to be grasped by the insertion tool without interference from the top and bottom arms;
a locking mechanism that holds the top clamping member and the bottom clamping member in the closed position wherein the locking member can be manually released to allow pivotal movement between the top and bottom clamping members;
wherein, when the locking mechanism is released, the top clamping member and the bottom clamping member can be pivoted between a closed position where the distal end of the top arm and the distal end of the bottom arm are proximate one another for holding the insertable medical device therebetween; and an open position where the top arm and the bottom arm are separated from one another in order to release the insertable medical device from the device carrier; and
wherein the device carrier further includes an insertable medical device having a head wherein the insertable medical device is held between the top arm and the bottom arm of the device; and
(B) an insertion tool comprising: a handle; an actuating lever; an actuating mechanism; and a collet; wherein the actuating lever is operably connected to the collet by the actuating mechanism so that movement of the lever causes the collet to grasp the insertable medical device; wherein the insertion tool further includes an alignment mechanism for orienting the insertion tool with respect to the device carrier.
